(19) United States
(12) Patent Application Publication
(10) Pub. No.: US 2024/0382637 A1
(43) Pub. Date: Nov. 21, 2024

(54) WRITING UTENSIL SANITIZING ASSEMBLY AND METHOD

(71) Applicant: Michael Stout, Southlake, TX (US)

(72) Inventor: Michael Stout, Southlake, TX (US)

(21) Appl. No.: 18/198,831

(22) Filed: May 17, 2023

(51) Int. Cl. A61L 2/26; A61L 2/00; A61L 2/18

(52) U.S. Cl. CPC A61L 2/26; A61L 2/0088; A61L 2/18; A61L 2202/122; A61L 2202/17

(57) ABSTRACT

A writing utensil sanitizing assembly and method for sanitizing a writing utensil prior to and after using the writing utensil to prevent the spread of germs and bacteria from touching the writing utensil includes a housing having a first end wall, a second end wall, and a perimeter wall attached to and extending between the first and second end walls. The first end wall has a first aperture extending therethrough, and the second end wall has a second aperture extending therethrough. The first and second apertures receive the writing utensil. An absorbent medium is positioned within the housing and has an open conduit extending therethrough and receives the writing utensil such that the writing utensil abuts a surface of the open conduit. The absorbent medium absorbs a sanitizing fluid for application onto the writing utensil when the writing utensil passes through the open conduit.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sanitizing device and more particularly pertains to a new sanitizing device for sanitizing a writing utensil prior to and/or after using the writing utensil to prevent the spread of germs and bacteria from touching the writing utensil.

### (2) Description of Related Art Including Information Disclosed Under 37 CFR 1.97 and 1.98

The prior art relates to sanitizing devices and teaches of sanitizing devices sterilizing an exterior surface of a writing utensil, but does not teach an efficient means for coating the exterior surface of the writing utensil with a sanitizing solution.

### BRIEF SUMMARY OF THE INVENTION

An embodiment of the disclosure meets the needs presented above by generally comprising a housing having a first end wall, a second end wall, and a perimeter wall attached to and extending between the first and second end walls. The first end wall has a first aperture extending therethrough, and the second end wall has a second aperture extending therethrough. The first and second apertures are axially aligned with each other and are configured to receive the writing utensil. An absorbent medium is positioned within the housing. The absorbent medium has an open conduit extending therethrough and is aligned with each of the first and second apertures. The open conduit is configured to receive the writing utensil such that the writing utensil abuts a surface of the open conduit. The absorbent medium is configured to absorb a sanitizing fluid for application onto the writing utensil when the writing utensil passed through the open conduit.

Another embodiment of the disclosure comprises a method including the steps of saturating an absorbent material with a sanitizing fluid and positioning the absorbent material within a housing. A writing utensil extends through a first aperture in a first end wall and a second aperture in a second end wall of the housing such that an outer surface of the writing utensil abuts the absorbent medium and is sanitized with the sanitizing fluid. The housing has a perimeter wall extending between the first and second end wall. The first and second apertures are axially aligned with each other. The absorbent medium has an open conduit extending therethrough and is aligned with each of the first and second apertures.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top isometric view of a writing utensil sanitizing assembly and method according to an embodiment of the disclosure.

FIG. 2 is an exploded view of an embodiment of the disclosure.

FIG. 3 is a top in-use view of an embodiment of the disclosure.

FIG. 4 is a side view of an embodiment of the disclosure.

FIG. 5 is a front view of an embodiment of the disclosure.

FIG. 6 is a bottom view of an embodiment of the disclosure.

FIG. 7 is a cross-sectional view of an embodiment of the disclosure taken along Line 7-7 of FIG. 6.

### DETAILED DESCRIPTION OF THE INVENTION

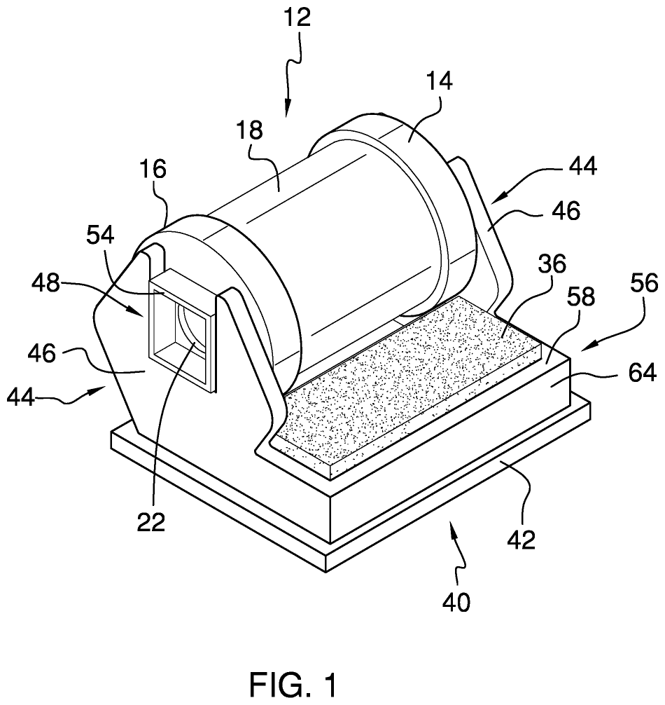

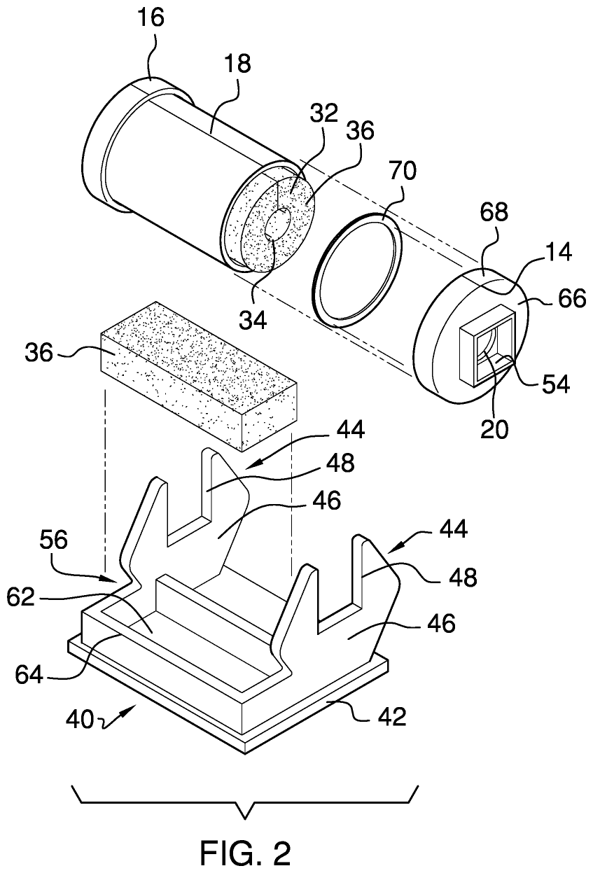

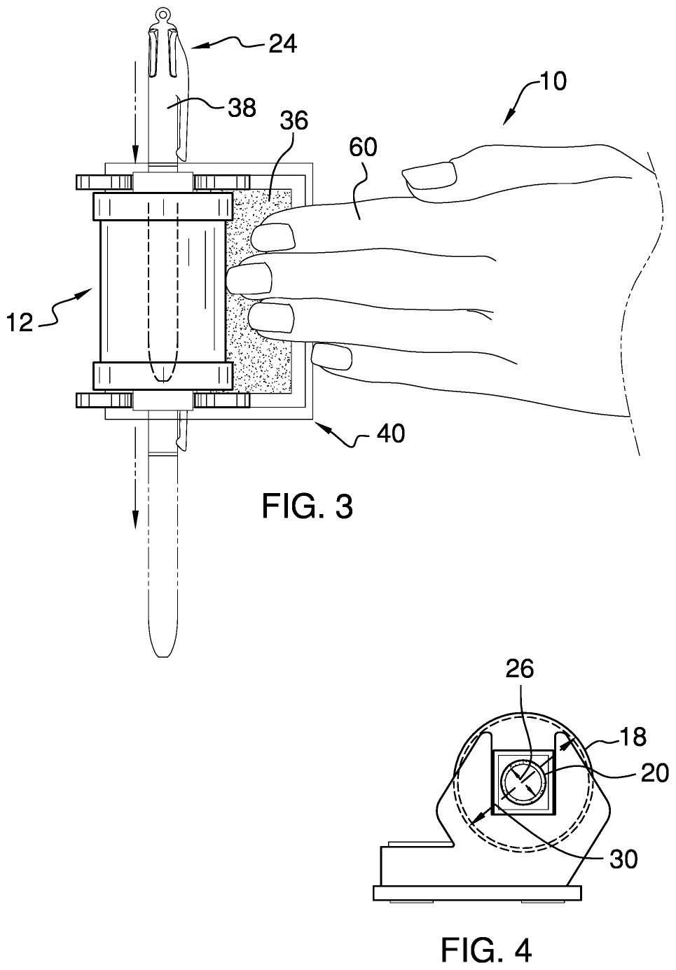

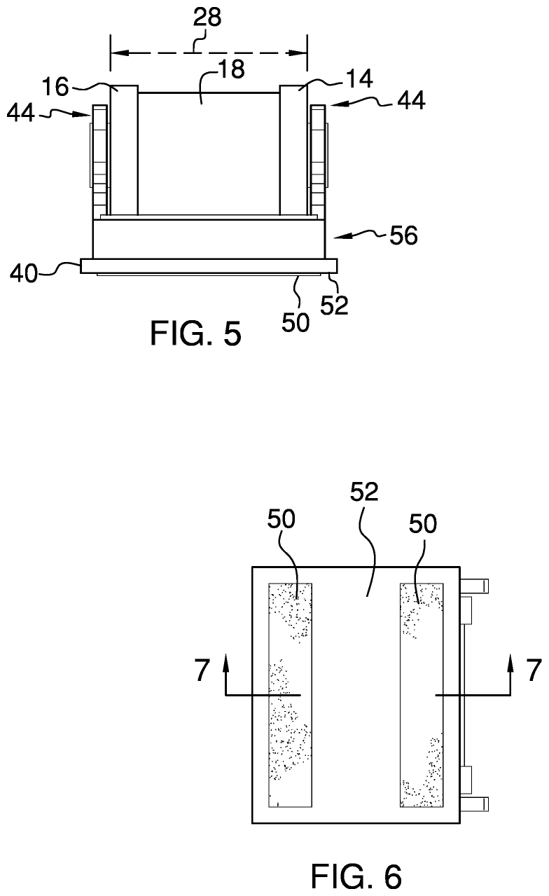

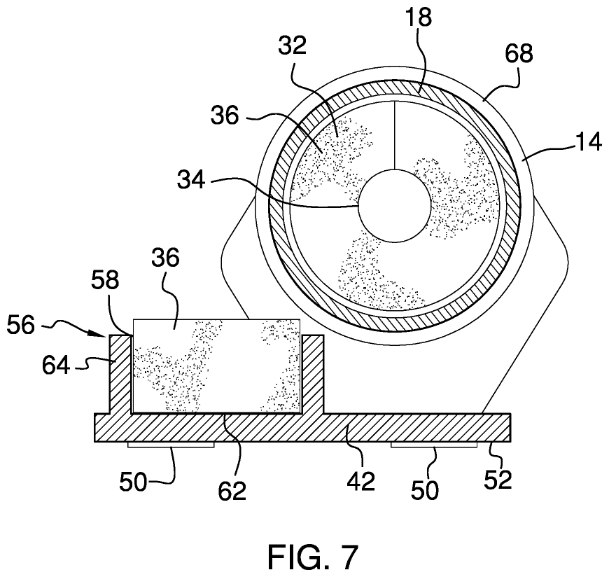

With reference now to the drawings, and in particular to FIGS. 1 through 7 thereof, a new sanitizing device embodying the principles and concepts of an embodiment of the disclosure and generally designated by the reference numeral 10 will be described.

As best illustrated in FIGS. 1 through 7, the writing utensil sanitizing assembly and method 10 generally comprises a housing 12 having a first end wall 14, a second end wall 16, and a perimeter wall 18 attached to and extending between the first 14 and second 16 end walls. The first end wall 14 has a first aperture extending therethrough, and the second end wall 16 has a second aperture 22 extending therethrough. The first 20 and second 22 apertures are axially aligned with each other and are configured to receive a writing utensil 24. The writing utensil 24 typically comprises a pen or a pencil. The first 20 and second 22 apertures may be configured to receive any elongated item such as a metal straw, a chop stick, or any other elongated item. The first 20 and second 22 apertures each have a circular shape and an inner diameter 26 being less than 0.75 inches. The housing 12 has a length 28 from the first end wall 14 to the second end wall 16 being between 1.0 inches and 4.0 inches. The perimeter wall 18 forms a cylinder having an outer diameter 30 being less than 2.0 inches.

An absorbent medium 32 is positioned within the housing 12. The absorbent medium 32 has an open conduit 34 extending therethrough and is aligned with each of the first 20 and second 22 apertures. The open conduit 34 is configured to receive the writing utensil 24 such that the writing utensil 24 abuts a surface of the open conduit 34. The absorbent medium 32 is configured to absorb a sanitizing fluid 36 for application onto the writing utensil 24 when the writing utensil 24 passed through the open conduit 34. The sanitizing fluid 36 sterilizes an exterior surface 38 of the writing utensil 24, wherein the writing utensil 24 is sterile immediately after exiting through the absorbent medium 32. The absorbent medium 32 normally comprises a sponge material and has a cylindrical shape corresponding with a tubular shape of the housing 12. The sponge material abuts the writing utensil 24 and therefore will also frictionally clean the writing utensil 24.

A mount 40 is engaged with the housing 12 and is configured to support the housing 12 when the writing utensil 24 is passed through the housing 12. The mount 40 includes a base 42 and a pair of supports 44 attached to and extending upwardly from the base 42. The pair of supports 44 releasably couple the housing 12 to the base 42. Each of the supports 44 includes a vertical wall 46 including a first mating member 48. An adhesive 50 is positioned on a bottom surface 52 of the base 42 and is configured to secure the base 42 to a planar surface. The adhesive 50 is typically a pressure sensitive adhesive 50, wherein the bottom surface 52 of the mount 40 is configured to be pressed against the planar surface.

Each of the first 14 and second 16 end walls has a respective one of a pair of second mating members 54 attached thereto, wherein the first mating member 48 of each support of the pair of supports 44 is engaged to one of the second mating members 54 of the pair of second mating members 54. Each of the second mating members 54 comprises a protrusion having an outer surface have a shape inhibiting rotation of the first 14 and second 16 end walls relative to the pair of supports 44. The first 20 and second 22 apertures extend through an associated one of the second mating members 54.

The first end wall 14 comprises a cap releasably engaged to the perimeter wall 18. The first end wall 14 includes an outer plate 66 and a peripheral shoulder 68 being attached to and being co-extensive with an exterior edge of the plate. The peripheral shoulder 68 extends over the perimeter wall 18 when the first end wall 14 is engaged with the perimeter wall 18. A seal 70 is positioned at a juncture of the first end wall 14 and the perimeter wall 18. The seal 70 typically comprises an O-ring continuously abutting against the absorbent medium 32 and the perimeter wall 18 of the housing 12. In another embodiment, the first 14 and second 16 end walls each may comprise a cap releasably engaged to the perimeter wall 18.

A trough 56 is attached to the mount 40 and extends laterally away therefrom. The trough 56 has an open upper side 58 and is configured to hold a quantity of sanitizing fluid 36, wherein the fingers 60 of the user are physically contactable with the sanitizing fluid 36 to sterilize the fingers 60 prior to or after inserting the writing utensil 24 through the absorbent medium 32. The trough 56 includes a bottom wall 62 being co-extensive and co-planar with the base 42. A peripheral wall 64 is attached to and extends upwardly from the bottom wall 62.

In use, the fingers 60 of the hand used to grip the writing utensil 24 are cleaned either before or after extending the writing utensil 24 through the first 20 and second 22 apertures by placing the fingers 60 in contact with sanitizing fluid 36 positioned in the trough 56. The absorbent material is saturated with the sanitizing fluid 36 and the absorbent material is positioned within the housing 12 thereafter. The writing utensil 24 extends through the first aperture in the first end wall 14 and the second aperture 22 in the second end wall 16 of the housing 12 such that the outer surface of the writing utensil 24 abuts the absorbent medium 32 and is sanitized with the sanitizing fluid 36.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A writing utensil sanitizing assembly configured for sanitizing an outer surface of a writing utensil, said assembly comprising:
a housing having a first end wall, a second end wall, and a perimeter wall being attached to and extending between said first and second end walls, said first end wall having a first aperture extending therethrough, said second end wall having a second aperture extending therethrough, said first and second apertures being axially aligned with each other and being configured to receive the writing utensil; and
an absorbent medium being positioned within said housing, said absorbent medium having an open conduit extending therethrough and being aligned with each of said first and second apertures, said open conduit being configured to receive said writing utensil such that the writing utensil abuts a surface of the open conduit, said absorbent medium being configured to absorb a sanitizing fluid for application onto the writing utensil when the writing utensil passed through said open conduit.

2. The writing utensil sanitizing assembly according to claim 1, further including a mount being engaged with the housing and being configured to support the housing when the writing utensil is passed through the housing.

3. The writing utensil sanitizing assembly according to claim 2, wherein said mount includes:
a base; and
a pair of supports being attached to and extending upwardly from said base, said pair of supports releasably coupling said housing to said base.

4. The writing utensil sanitizing assembly according to claim 3, further including an adhesive being positioned on a bottom surface of said base and being configured to secure said base to a planar surface.

5. The writing utensil sanitizing assembly according to claim 3, wherein each of said supports includes a vertical wall including a first mating member.

6. The writing utensil sanitizing assembly according to claim 5, further including a pair of second mating members, each of said first and second end walls having one of said second mating members attached thereto, wherein the first mating member of each support of said pair of supports is engaged to one of the second mating members of the pair of second mating members.

7. The writing utensil sanitizing assembly according to claim 6, each of said second mating members comprises a protrusion having an outer surface having a shape inhibiting rotation of said first and second end walls relative to said pair of supports.

8. The writing utensil sanitizing assembly according to claim 3, further including a trough being attached to said mount and extending laterally away therefrom, said trough having an open upper side and being configured to hold a quantity of sanitizing fluid, said trough including a bottom wall being co-extensive and co-planar with said base, a peripheral wall being attached to and extending upwardly from said bottom wall.

9. The writing utensil sanitizing assembly according to claim 1, wherein said first and second apertures each having a circular shape and a diameter being less than 0.75 inches, said housing having a length from said first end wall to said second end wall being between 1.0 inches and 4.0 inches.

10. The writing utensil sanitizing assembly according to claim 1, wherein said absorbent medium comprising a sponge material.

11. The writing utensil sanitizing assembly according to claim 1, wherein said first end wall comprises a cap releasably engaged to said perimeter wall, said first end wall including an outer plate and a peripheral shoulder being attached to an being co-extensive with an exterior edge of said plate, said peripheral shoulder extending over said perimeter wall when said first end wall is engaged with said perimeter wall.

12. The writing utensil sanitizing assembly according to claim 10, further including a seal being positioned at a juncture of said first end wall and said perimeter wall.

13. A writing utensil sanitizing assembly configured for sanitizing an outer surface of a writing utensil, said assembly comprising:
a housing having a first end wall, a second end wall, and a perimeter wall being attached to and extending between said first and second end walls, said first end wall having a first aperture extending therethrough, said second end wall having a second aperture extending therethrough, said first and second apertures being axially aligned with each other and being configured to receive the writing utensil;
an absorbent medium being positioned within said housing, said absorbent medium having an open conduit extending therethrough and being aligned with each of said first and second apertures, said open conduit being configured to receive said writing utensil such that the writing utensil abuts a surface of the open conduit, said absorbent medium being configured to absorb a sanitizing fluid for application onto the writing utensil when the writing utensil passed through said open conduit;
a mount is engaged with the housing and is configured to support the housing when the writing utensil is passed through the housing, said mount including:
a base;
a pair of supports being attached to and extending upwardly from said base, said pair of supports releasably coupling said housing to said base, each of said supports including a vertical wall including a first mating member;
an adhesive being positioned on a bottom surface of said base and being configured to secure said base to a planar surface;
a pair of second mating members, each of said first and second end walls having one of said second mating members attached thereto, wherein the first mating member of each support of said pair of supports is engaged to one of the second mating members of the pair of second mating members, each of said second mating members comprises a protrusion having an outer surface having a shape inhibiting rotation of said first and second end walls relative to said pair of supports, said first and second apertures extending through an associated one of said second mating members;
a trough being attached to said mount and extending laterally away therefrom, said trough having an open upper side and being configured to hold a quantity of sanitizing fluid, said trough including a bottom wall being co-extensive and co-planar with said base, a peripheral wall being attached to and extending upwardly from said bottom wall;
said first and second apertures each having a circular shape and a diameter being less than 0.75 inches, said housing having a length from said first end wall to said second end wall being between 1.0 inches and 4.0 inches, said perimeter wall forming a cylinder having a diameter being less than 2.0 inches;
said absorbent medium comprising a sponge material;
said absorbent medium having a cylindrical shape;
said first end wall comprising a cap releasably engaged to said perimeter wall, said first end wall including an outer plate and a peripheral shoulder being attached to an being co-extensive with an exterior edge of said plate, said peripheral shoulder extending over said perimeter wall when said first end wall is engaged with said perimeter wall;
a seal being positioned at a juncture of said first end wall and said perimeter wall.

14. A method of sanitizing an outer surface of a writing utensil including the step of:
saturating an absorbent material with a sanitizing fluid and positioning the absorbent material within a housing;
extending a writing utensil through a first aperture in a first end wall and a second aperture in a second end wall of the housing such that an outer surface of the writing utensil abuts the absorbent medium and is sanitized with the sanitizing fluid, the housing having a perimeter wall extending between the first and second end wall, said first and second apertures being axially aligned with each other, said absorbent medium having an open conduit extending therethrough and being aligned with each of said first and second apertures.

15. The method of sanitizing an outer surface of a writing utensil as in claim 14, further including the step of:
cleaning the fingers of a hand used to grip the writing utensil either before or after extending the writing utensil through the first and second apertures, said step of cleaning the fingers including placing the fingers in contact with sanitizing fluid positioned in a trough, the trough being attached to a mount holding the housing.
